(19) United States
(12) Patent Application Publication
(10) Pub. No.: US 2023/0233547 A1
(43) Pub. Date: Jul. 27, 2023

(54) PHARMACEUTOCAL COMPOSITION CONTAINING N-(4-(6,7-DIMETHOXYQUINOLIN-4-YLOXY)PHENYL)-N?-(4-FLUOROPHENLY)CYCLOPROPANE-1,1-DICARBOXAMIDE, (2S)-HYDROXYBUTANEDIOATE

(71) Applicant: MSN Laboratories Private Limited, R & D Center, Hyderabad, Telangana (IN)

(72) Inventors: Thirumalai Rajan SRINIVASAN, Hyderabad, Telangana (IN); Eswaraiah SAJJA, Hyderabad, Telangana (IN); Nagaraju CH V S, Hyderabad, Telangana (IN); Ramachandra Reddy POREDDY, Hyderabad, Telangana (IN); Srinivasulu POLYSETTY, Hyderabad, Telangana (IN); Raghava Raju Sudhakar VALIVARTHI, Hyderabad, Telangana (IN)

(73) Assignee: MSN Laboratories Private Limited, R & D Center, Hyderabad, Telangana (IN)

(21) Appl. No.: 17/918,911

(22) PCT Filed: Apr. 15, 2021
(86) PCT No.: PCT/IB2021/053107

§ 371 Date: Oct. 14, 2022

(30) Foreign Application Priority Data
Apr. 14, 2020 (IN) 202041016104

(51) Int. Cl. A61K 31/47; C07D 215/233

(52) U.S. Cl. CPC A61K 31/47; C07D 215/233

(57) ABSTRACT

The present invention relates to a pharmaceutical composition comprising N-(4-(6,7-dimethoxy quinolin-4-yloxy)phenyl)-N′-(4-fluoro phenyl) cyclopropane-1,1-dicarboxamide, (2S)-hydroxy butanedioate of formula-1

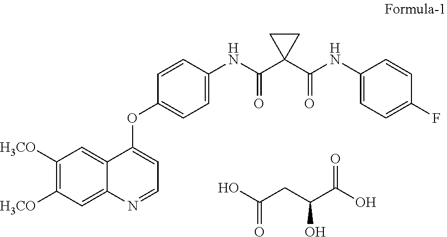 [SMILES: COc1cc2nccc(Oc3ccc(NC(=O)C4(C(=O)Nc5ccc(F)cc5)CC4)cc3)c2cc1OC ; O=C(O)C[C@H](O)C(=O)O]

and a pharmaceutically acceptable carrier wherein the said pharmaceutical composition has a dimer impurity of formula-2 less than about 2.0% as measured by HPLC.

### RELATED APPLICATION

This application claims the benefit of priority of our Indian patent application number 202041016104 filed on 14 Apr. 2020 which is incorporated herein by reference.

### FIELD OF THE INVENTION

The present invention relates to a pharmaceutical composition comprising N-(4-(6,7-dimethoxyquinolin-4-yloxy)phenyl)-N′-(4-fluorophenyl)cyclopropane-1,1-dicarboxamide, (2S)-hydroxybutanedioate of formula-1 having a dimer impurity, (Z)-4-((4-((6,7-dimethoxy quinolin-4-yl)oxy)phenyl)amino)-3-((4-fluorophenyl)carbamoyl)-4-oxobutyl N-(4-((6,7-dimethoxyquinolin-4-yl)oxy)phenyl)-1-((4-fluorophenyl)carbamoyl)cyclopropanecarbimidate of formula-2 less than about 2.0% as measured by HPLC.

### BACKGROUND OF THE INVENTION

Cabozantinib (S)-malate, chemically known as N-(4-(6,7-dimethoxyquinolin-4-yloxy) phenyl)-N′-(4-fluorophenyl)cyclopropane-1,1-dicarboxamide, (2S)-hydroxybutanedioate and has the following chemical structure.

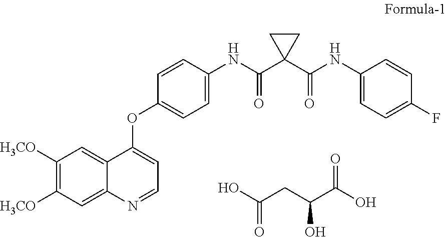 [SMILES: COc1cc2nccc(Oc3ccc(NC(=O)C4(C(=O)Nc5ccc(F)cc5)CC4)cc3)c2cc1OC ; O=C(O)C[C@H](O)C(=O)O]

Cabozantinib (S)-malate is marketed under the trade name of COMETRIQ and CABOMETYX by Exelixis, Inc. COMETRIQ is indicated for the treatment of patients with progressive, metastatic medullary thyroid cancer. CABOMETYX is a kinase inhibitor indicated for the renal cell carcinoma (RCC) and in combination with nivolumab, is indicated for the first-line treatment of patients with advanced RCC. COMETRIQ capsules contain 20 mg and 80 mg of Cabozantinib as active ingredient and CABOMETYX tablets contain 60 mg, 40 mg and 20 mg of Cabozantinib as active ingredient. WO2012009722 discloses tablet composition of Cabozantinib and WO2012109510 discloses capsule tablet composition of Cabozantinib.

In the pharmaceutical industry, there is a constant need to work on identifying different pharmaceutical compositions that positively affect the drug's dissolution profile, bioavailability, bioequivalence, stability, etc., which all play important roles in determining a drug's market acceptance and success.

For example, Cabozantinib is subject to structural modifications by a variety of degradation mechanisms resulting in problems of chemical and physical instability of the formulation. Hence, there is a need for the development of pharmaceutical compositions with improved stability, excellent storage, and handling stabilities, bioavailability, etc.

### DETAILED DESCRIPTION OF THE INVENTION

The present invention provides a stable pharmaceutical composition of N-(4-(6,7-dimethoxyquinolin-4-yloxy)phenyl)-N′-(4-fluorophenyl)cyclopropane-1,1-dicarboxamide, (2S)-hydroxybutanedioate of formula-1.

The present invention relates to a pharmaceutical composition comprising N-(4-(6,7-dimethoxyquinolin-4-yloxy)phenyl)-N′-(4-fluorophenyl)cyclopropane-1,1-dicarboxamide, (2S)-hydroxybutanedioate of formula-1 and a pharmaceutically acceptable carrier

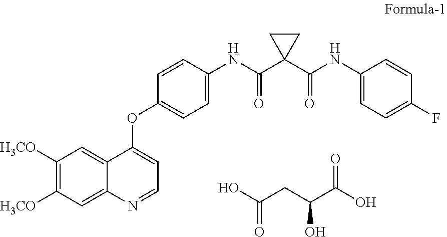 [SMILES: COc1cc2nccc(Oc3ccc(NC(=O)C4(C(=O)Nc5ccc(F)cc5)CC4)cc3)c2cc1OC ; O=C(O)C[C@H](O)C(=O)O]

wherein the said pharmaceutical composition has a dimer impurity of formula-2 less than about 2.0% as measured by HPLC.

The present invention relates to a pharmaceutical composition comprising crystalline form of N-(4-(6,7-dimethoxyquinolin-4-yloxy)phenyl)-N′-(4-fluorophenyl)cyclopropane-1,1-dicarboxamide, (2S)-hydroxybutanedioate of formula-1 and a pharmaceutically acceptable carrier wherein the said pharmaceutical composition has a dimer impurity of formula-2 less than about 2.0% as measured by HPLC.

The present invention further relates to a pharmaceutical composition comprising crystalline form-S of N-(4-(6,7-dimethoxyquinolin-4-yloxy)phenyl)-N′-(4-fluorophenyl) cyclopropane-1,1-dicarboxamide, (2S)-hydroxybutanedioate of formula-1 and a pharmaceutically acceptable carrier wherein the said pharmaceutical composition has a dimer impurity of formula-2 less than about 2.0% as measured by HPLC. The crystalline form-S of N-(4-(6,7-dimethoxyquinolin-4-yloxy)phenyl)-N′-(4-fluorophenyl)cyclopropane-1,1-dicarboxamide, (2S)-hydroxybutanedioate of formula-1 and its preparation is described, for example in WO2018/104954, the descriptions of which are incorporated herein by reference and form part of the disclosure in the present application.

The present application identifies challenges in developing pharmaceutical formulations comprising compound of Formula-1. Specifically compound of Formula-1 can degrade to form a compound having a dimer impurity (referred to herein as Formula-2). Reducing conversion of the drug substance into its dimer-containing degradation product is desirable, for example, to meet the required pharmaceutical specifications of the drug product. Thus, it has been determined in the present application that a pharmaceutical composition reduces generation of the dimer degradation product (i.e., Formula-2).

In an aspect of the invention, the pharmaceutical compositions as described here, in particular in the form of a capsule or a tablet, comprise a therapeutically effective amount of N-(4-(6,7-dimethoxyquinolin-4-yloxy)phenyl)-N′-(4-fluorophenyl)cyclopropane-1,1-dicarboxamide, (2S)-hydroxybutanedioate of formula-1.

The present invention provides for pharmaceutical compositions comprising as active pharmaceutical ingredient N-(4-(6,7-dimethoxyquinolin-4-yloxy)phenyl)-N′-(4-fluorophenyl) cyclopropane-1,1-dicarboxamide, (2S)-hydroxybutanedioate of formula-1 with an improved stability or a longer shelf life. The shelf life of the compositions of the present invention is at least 12 months, at least 18 months or at least 24 months wherein the said pharmaceutical composition has a dimer impurity of formula-2 less than about 2.0% as measured by HPLC.

The present invention relates to a pharmaceutical composition comprising N-(4-(6,7-dimethoxyquinolin-4-yloxy)phenyl)-N′-(4-fluorophenyl)cyclopropane-1,1-dicarboxamide, (2S)-hydroxybutanedioate of formula-1

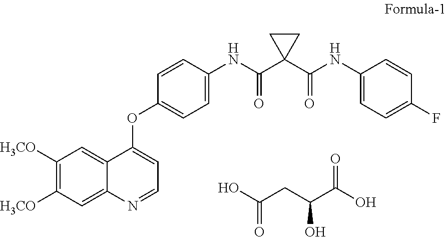 [SMILES: COc1cc2nccc(Oc3ccc(NC(=O)C4(C(=O)Nc5ccc(F)cc5)CC4)cc3)c2cc1OC ; O=C(O)C[C@H](O)C(=O)O]

and a pharmaceutically acceptable carrier wherein the said pharmaceutical composition has a dimer impurity of formula-2 less than about 2% or less than about 1.5% or less than about 1% or less than about 0.5% or less than about 0.1% or less than about 0.05% as measured by HPLC.

The said dimer impurity is represented by following structure:

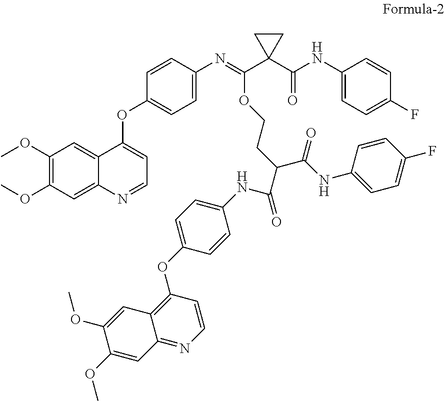 [SMILES: COc1cc2nccc(Oc3ccc(/N=C(\OCCC(C(=O)Nc4ccc(F)cc4)C(=O)Nc4ccc(Oc5ccnc6cc(OC)c(OC)cc56)cc4)C4(C(=O)Nc5ccc(F)cc5)CC4)cc3)c2cc1OC]

In an aspect of the invention, the pharmaceutical compositions as described herein are suitable for oral administration, such as capsules or tablets, a pharmaceutical composition in the form of a tablet in particular for oral administration being preferred.

The impurities can be identified by analytical techniques such as chromatographic and mass spectrometry techniques among others. For example, HPLC, GC, or other chromatography methods can be used to identify said impurity.

### HPLC Method of Analysis

Cabozantinib (S)-malate and its related substances were analyzed by HPLC with the following chromatographic conditions:
Apparatus: Waters HPLC with UV Detector (or) Equivalent. Column: Unison UK C18,150 x 4.6 mm, 3 μm; Wavelength: 245 nm; Column temperature: 25° C.; Injection volume: 10 μL;
Elution: Gradient;
Buffer Preparation: Weigh accurately and transfer about 2.75 mg of potassium di hydrogen orthophosphate in 1000 ml of milli-Q water, add 1 ml triethyl amine and mix. Adjust pH with dilute ortho-phosphoric acid solution and mix.
Mobile phase-A: Buffer 100%;
Mobile phase-B: Acetonitrile: Methanol: water

As used herein, abbreviations for methods and techniques may include: High-performance Liquid Chromatography (HPLC), Ultra Performance Liquid Chromatography (UPLC), Supercritical fluid chromatography (SFC), Gas chromatography (GC), Good Manufacturing Practices (GMP), Current Good Manufacturing Practices (cGMP), not more than (NMT), and not less than (NLT).

High purity of a preparation meets both of the following two criteria: 1) the overall level of purity is at least about 97%; that is, the desired product (N-(4-(6,7-dimethoxyquinolin-4-yloxy)phenyl)-N′-(4-fluorophenyl)cyclopropane-1,1-dicarboxamide, (2S)-hydroxybutanedioate) accounts for at least 97% of the preparation; and 2) any individual dimer-related impurity of formula-2 is present in an amount of less than about 2.0% of the preparation. The purity is preferably measured by HPLC analysis.

The tablets of the invention can be produced by conventional tabletting techniques together with pharmaceutically acceptable excipients (pharmaceutically acceptable carrier) and with conventional tabletting machines. As known in the art, tablet blends may be dry-granulated or wet-granulated before tabletting. It will be appreciated that the person skilled in the art will be able to recognize the most appropriate way to manufacture the compositions of the present invention. Among these, wet granulation methods are preferably used for the production, from a standpoint of being able to uniformly mix the active ingredient and other components in the tablet, and being able to obtain a tablet whose components are uniformly distributed therein.

In order to facilitate the swallowing of such a pharmaceutical composition for oral administration by a mammal, in particular a human, it is advantageous to give the compositions, in particular tablets, an appropriate shape.

As used herein, the term “pharmaceutical composition” or “pharmaceutical formulations” include tablets, pills, powders, liquids, suspensions, emulsions, granules, capsules, suppositories, or injection preparations.

Examples of pharmaceutically acceptable carriers are filler, binder, disintegrant, glidant, and lubricant, and optionally a film coating material. Fillers, binders, disintegrants, glidants, lubricants and film coating materials are set forth below and are described in more detail in the Handbook of Pharmaceutical Excipients, Second Edition, Ed. A. Wade and P. J. Weller, 1994, The Pharmaceutical Press, London, England.

The term carrier or excipient as used herein refers to inert materials which impart satisfactory processing and compression characteristics into the formulation or imparts desired physical characteristics to the finished product.

A pharmaceutical composition of the present invention is a tablet comprising compound of formula-1 and excipients selected from the group comprising of filler, binder, disintegrant, glidant and lubricant. Optionally, it may be coated or uncoated.

The present invention provides stable pharmaceutical composition comprising;
a) the compound of formula-1
b) a filler
c) a disintegrant
d) a surfactant
e) a glidant and
f) a lubricant.

In an embodiment of the present invention, a pharmaceutical composition containing compound of formula-1 comprises a filler. A variety of materials may be used as diluents or fillers. Examples are lactose monohydrate, anhydrous lactose, sucrose, dextrose, mannitol, sorbitol, starch, Granulated corn starch, cellulose (e.g. micro-crystalline cellulose (Avicel™), silicified microcrystalline cellulose), dihydrated or anhydrous dibasic calcium phosphate, and others known in the art, and mixtures thereof (e.g. spray-dried mixture of lactose monohydrate (75%) with microcrystalline cellulose (25%) which is commercially available as Microcelac™). The total amount of diluent or filler in the pharmaceutical compositions of the present invention may conveniently range from about 10% to about 95% w/w and preferably ranges from about 20% to about 90% w/w, or from about 20% to about 85% w/w.

Binders can be employed in the pharmaceutical compositions of the present invention. Suitable binders are water-soluble polymers, such as alkylcelluloses such as methylcellulose; hydroxyalkylcelluloses such as hydroxymethylcellulose, hydroxyethylcellulose, hydroxyl propylcellulose and hydroxybutylcellulose; hydroxyalkyl alkylcelluloses such as hydroxyl ethylmethylcellulose and hydroxypropyl methylcellulose; carboxyalkylcelluloses such as carboxymethylcellulose; alkali metal salts of carboxyalkylcelluloses such as sodium carboxymethylcellulose; carboxyalkylalkylcelluloses such as carboxymethylethylcellulose; carboxyalkylcellulose esters; starches; pectines such as sodium carboxymethylamylopectine; chitin derivates such as chitosan; di-, oligo- and polysaccharides such as trehalose, cyclodextrins and derivatives thereof, alginic acid, alkali metal and ammonium salts thereof, carrageenans, galactomannans, tragacanth, agar-agar, gummi arabicum, guar gummi and xanthan gummi; polyacrylic acids and the salts thereof; polymethacrylic acids, the salts and esters thereof, methacrylate copolymers; polyvinylpyrrolidone (PVP), polyvinylalcohol (PVA) and copolymers thereof, e.g. PVP-VA. Preferably, the water-soluble polymer is a hydroxyalkyl alkylcelluloses, such as for example hydroxypropylmethyl cellulose, e.g. hydroxypropylmethyl cellulose 15 cps. The total amount of binder in the pharmaceutical compositions of the present invention may conveniently range from about 0.1 to 20% by weight with respect to the weight of the tablet (when the tablet is coated, the weight of the uncoated tablet), and more preferably about 0.5 to 5% by weight.

In an embodiment of the present invention, a pharmaceutical composition containing compound of formula-1 comprises a disintegrant. A disintegrant is a substance or a mixture of substances added to facilitate breakup or disintegrate after administration. The disintegrant may be any pharmaceutically acceptable disintegrant available in the tabletting art, including alginic acid, carboxymethylcellulose calcium, carboxymethylcellulose sodium, colloidal silicon dioxide, croscarmellose sodium, crospovidone, guargum, magnesium aluminum silicate, methylcellulose, microcrystalline cellulose, polyacrilin potassium, powdered cellulose, pregelatinized starch, sodium alginate, starch, and the like, or mixtures thereof. The total amount of disintegrant in the pharmaceutical compositions of the present invention may conveniently range from about 0.1 to 20% by weight with respect to the weight of the tablet (when the tablet is coated, the weight of the uncoated tablet), and more preferably about 0.5 to 10% by weight.

In an embodiment of the present invention, a pharmaceutical composition containing compound of formula-1 comprises a glidant. The glidant may be any pharmaceutically acceptable glidant which contributes to the compressibility, flowability, and homogeneity of the formulation and which minimizes segregation and does not significantly interfere with the release mechanism of the binders as set forth above. Preferably, the glidant is selected to improve the flow of the formulation.

In an embodiment of the present invention, a pharmaceutical composition containing compound of formula-1 comprises a lubricant. Lubricants are employed to prevent adhesion of the tablet material to the surface of dyes and punches. The lubricant may be any pharmaceutically acceptable lubricant which substantially prevents segregation of the powder by contributing to homogeneity of the formulation and which exhibits good flowability. Preferably, the lubricant functions to facilitate compression of the tablets and ejection of the tablets from the die cavity. Such lubricants may be hydrophilic or hydrophobic, and examples include magnesium stearate, lubritab (R., Stearic acid, talc, and other lubricants known in the art or to be developed which exhibit acceptable or comparable properties, or mixtures thereof. Examples of lubricants include calcium stearate, glyceryl monostearate, glyceryl palmitostearate, hydrogenated castor oil, hydrogenated vegetable oil, light mineral oil, magnesium stearate, mineral oil, polyethylene glycol, sodium benzoate, sodium lauryl sulfate, sodium stearyl fumarate, stearic acid, talc, zinc stearate, and the like, or mixtures thereof. The lubricant content is not particularly limited, and is preferably about 0.1 to 10% by weight with respect to the weight of the tablet (when the tablet is coated, the weight of the uncoated tablet), more preferably about 0.2 to 7% by weight, and still more preferably about 0.3 to 5% by weight.

In an embodiment of the present invention, a pharmaceutical composition containing compound of formula-1 comprises an optional film coating. The film coat concentration can be about 1 to about 10 percent by weight on a solid basis of the directly compressible formulation. Film coating suspensions may include combinations of the following components: hypromeollose, carboxymethylcellulose sodium, carnauba wax, cellulose acetate phthalate, acetyl alcohol, confectioner's sugar, ethyl cellulose, gelatin, hydroxy ethyl cellulose, hydroxypropyl cellulose, hydroxypropyl methylcellulose, liquid glucose, maltodextrin, methyl cellulose, microcrystalline wax, Opadry and Opadry II, poly methacrylates, polyvinyl alcohol, shellac, sucrose, talc, titanium dioxide, and zein.

The present invention provides stable pharmaceutical composition comprising;
a) the compound of formula I, in a total amount of up to 50% by weight based on the total weight of the pharmaceutical composition,
b) a mixture of at least one or more filler in a total amount of 20 to 85% by weight based on the total weight of the pharmaceutical composition,
c) a binder in a total amount of 0.5 to 5% by weight based on the total weight of the pharmaceutical composition
d) a disintegrant in a total amount of 0.5-10% by weight based on the total weight of the pharmaceutical composition,
e) a lubricant.

The present invention relates to a stable pharmaceutical composition comprising:
a) the compound of formula I, in a total amount of up to 50% by weight based on the total weight of the pharmaceutical composition,
b) a mixture of at least one or more filler in a total amount of 20 to 85% by weight based on the total weight of the pharmaceutical composition,
c) a binder in a total amount of 0.5 to 5% by weight in weight based on the total weight of the pharmaceutical composition
d) a disintegrant in a total amount of 0.5-10% by weight based on the total weight of the pharmaceutical composition,
e) a lubricant.
   wherein the said pharmaceutical composition has a dimer impurity of formula-2 less than about 2.0% as measured by HPLC

The present invention relates to a process for preparing stable pharmaceutical composition comprising: Compound (I), one or more filler, disintegrant were weighed and mixed. A separately prepared aqueous solution of binder was added to the powder mixture, followed by wet kneading granulation. After drying and sizing the resulting granules, extra granular ingredients and the lubricant was added thereto and mixed. The final blend is then compressed on a tablet machine to obtain tablet cores. After the manufacturing of the tablet cores, the cores are film coated.

1-10. (canceled)

11. A compound (Z)-4-((4-((6,7-dimethoxy quinolin-4-yl)oxy)phenyl)amino)-3-((4-fluorophenyl)carbamoyl)-4-oxobutyl N-(4-((6,7-dimethoxyquinolin-4-yl)oxy)phenyl)-1-((4-fluorophenyl)carbamoyl)cyclopropanecarbimidate of formula-2:

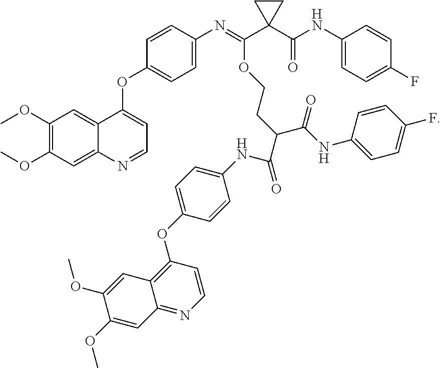 [SMILES: COc1cc2nccc(Oc3ccc(/N=C(\OCCC(C(=O)Nc4ccc(F)cc4)C(=O)Nc4ccc(Oc5ccnc6cc(OC)c(OC)cc56)cc4)C4(C(=O)Nc5ccc(F)cc5)CC4)cc3)c2cc1OC]

12. A pharmaceutical composition comprising N-(4-(6,7-dimethoxyquinolin-4-yloxy) phenyl)-N′-(4-fluorophenyl)cyclopropane-1,1-dicarboxamide, (2S)-hydroxybutanedioate of formula-1 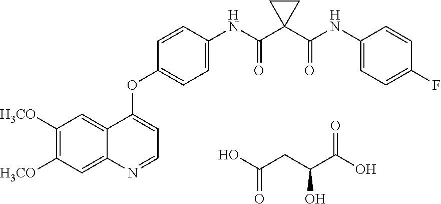 [SMILES: COc1cc2nccc(Oc3ccc(NC(=O)C4(C(=O)Nc5ccc(F)cc5)CC4)cc3)c2cc1OC ; O=C(O)C[C@H](O)C(=O)O]
and a pharmaceutically acceptable carrier wherein the said pharmaceutical composition has a dimer impurity of formula-2 less than about 2.0% as measured by HPLC.

13. A pharmaceutical composition according to claim 12, wherein the dimer impurity of formula-2 is less than about 1% as measured by HPLC.

14. A pharmaceutical composition according to claim 12, wherein the dimer impurity of formula-2 is less than about 0.5% as measured by HPLC.

15. A pharmaceutical composition according to claim 12, wherein the dimer impurity of formula-2 is less than about 0.1% as measured by HPLC.

16. A pharmaceutical composition according to claim 12, wherein the N-(4-(6,7-dimethoxyquinolin-4-yloxy)phenyl)-N-40 -(4-fluorophenyl)cyclopropane-1,1-dicarboxamide, (2S)-hydroxybutanedioate of formula-1 is in crystalline form.

17. A pharmaceutical composition comprising crystalline form of formula-1 according to claim 16 is crystalline form-S.

18. A pharmaceutical composition according to claim 12, wherein the pharmaceutical composition is a tablet or capsule.

19. A pharmaceutical composition according to claim 18, wherein the pharmaceutical composition is a coated tablet.

20. A pharmaceutical composition comprising:
a) the compound of formula-1, in a total amount of up to 50% by weight based on the total weight of the pharmaceutical composition,
wherein said pharmaceutical composition has less than about 2.0% of dimer impurity of formula-2 as measured by HPLC.

21. A pharmaceutical composition according to claim 20, further comprising:
a) a mixture of at least one or more filler in a total amount of 20% to 85% by weight based on the total weight of the pharmaceutical composition,
b) a binder in a total amount of 0.5% to 5% by weight based on the total weight of the pharmaceutical composition,
c) a disintegrant in a total amount of 0.5% to 10% by weight based on the total weight of the pharmaceutical composition, and
d) a lubricant.

22. A pharmaceutical composition according to claim 19, wherein the pharmaceutical composition is obtained by direct compression, wet granulation, or dry granulation.

23. A method of treating cancer in a subject in need thereof comprising administering to the subject a pharmaceutical composition according to claim 12.
